(19) United States
(12) Patent Application Publication
(10) Pub. No.: US 2002/0104545 A1
(43) Pub. Date: Aug. 8, 2002

(54) Earphone with coverings

(76) Inventor: Hsi Kuang Ma, Taipei (TW)

Correspondence Address: Dougherty & Troxell, ONE SKYLINE PLACE, 5205 LEESBURG PIKE, SUITE 1404, FALLS CHURCH, VA, 22041, US

(21) Appl. No.: 09/788,379

(22) Filed: Feb. 21, 2001

(30) Foreign Application Priority Data
Feb. 5, 2001 (TW) 90201749

(51) Int. Cl. A61F 11/00

(52) U.S. Cl. 128/864; 128/868

(57) ABSTRACT

An earphone with coverings comprises two opposite coverings and a covering connection. The covering connection connects with these two opposite coverings at both ends thereof. Each covering provides current leads for transmitting signals. A projection extends outward from a lateral side of each covering facing the corresponding ear for plugging into the ears during the earphone being worn. The projection has a size corresponding to the external acoustic meatus of the respective ear and provides at least a sound opening so as to transmit the sound into the meatus.

### BACKGROUND OF THE INVENTION

1. Field of The Invention

The present invention relates to an earphone with coverings, and particularly to an improvement of earphone with coverings.

2. Description of Related Art

The earphone has been utilized for communicating information, receiving broadcast, and listening to stereo for years.

The conventional earphone with coverings as shown in FIG. 1 provides two opposite ear coverings facing to each other and each covering has an approximately flat shape. That is, the voice emitting components are placed in the coverings with the flat sides thereof being arranged to facially touch the external ears respectively while the earphone is worn.

A disadvantage of conventional earphone is that the coverings thereof press against the external ears, which have a shape of irregularity and abruptness, and cause part of the external ears to subject discomfort. Moreover, the voice emitting components in the coverings are farther away the ears such that the sound is unable to enter the ears completely and part of sound may loss during transmitting to the ears. In addition, the foreign noise may mix with the sound to result in a undesirable effect of sound listening.

### SUMMARY OF THE INVENTION

An object of the present invention is to provide an earphone with coverings, which extends a projecting from the coverings thereof for being able to plug into the ears respectively so as to enhance the sound effect and to avoid the interference of foreign noise.

### BRIEF DESCRIPTION OF THE DRAWINGS

The present invention can be more fully understood by referring to the following description and accompanying drawings, in which:

FIG. 1 is a plan view of conventional earphone with covering; and

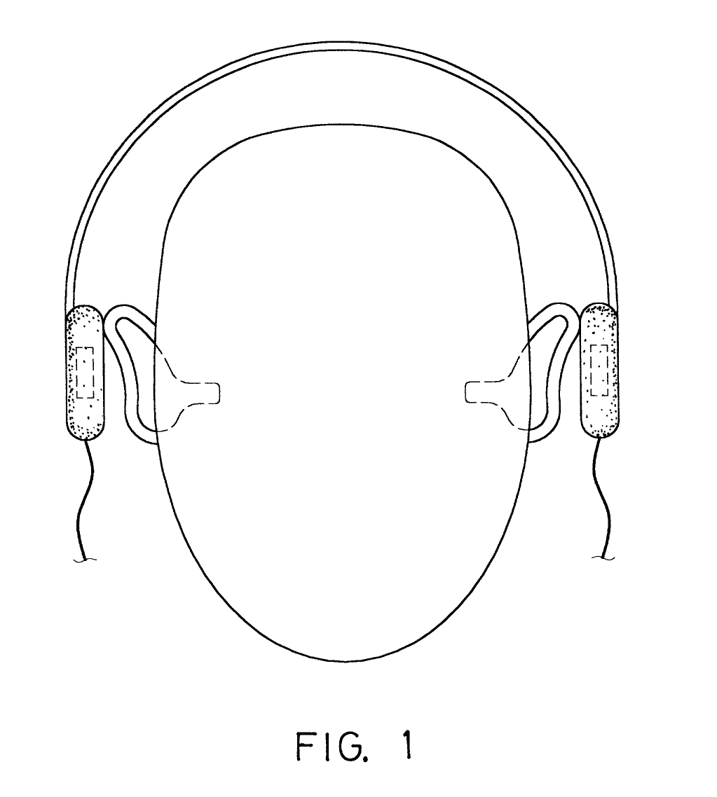

FIG. 2 is a plan view of earphone with covering according to the present invention.

### DETAILED DESCRIPTION OF THE PREFERRED EMBODIMENTS

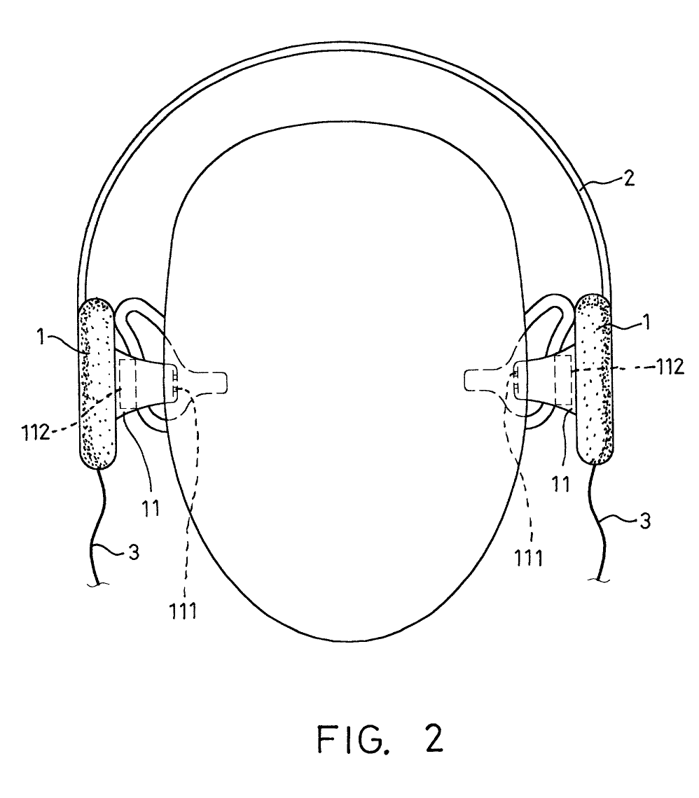

Referring to FIG. 2, the earphone of present invention basically comprises two coverings 1, and a covering connection 2, and lead wires 3.

Each one of coverings 1 is solid with soft pad surrounding the border thereof and the inner lateral side thereof, i.e., the lateral side facing the ear corresponding thereto during wearing the earphone extends a projection 11. The projection 11 has a cross section not greater than the external acoustic meatus so as to be plugged into the external acoustic meatus while the earphone is worn. The projection 11 has an end and at least a sound opening 111 is arranged surrounding the end at the rim thereof and multiple sound openings 111 can be arranged in case of the size of opening is smaller. The purpose of sound opening 111 is for sound output. Besides, in order to have the sound output enter the ear directly and effectively, voice emitting component 112, i.e., the loudspeaker can be disposed as closer to the end rim of projection 11 as possible.

The covering connection 2 at both ends thereof joins the coverings 1 adjustably or fixedly as the conventional earphone does. Because these are prior art, no detail will be described further.

Referring FIG. 2 again, the current leads 3, which are signal lines, and an end thereof connects with the earphone parts in the coverings 1 respectively and the other end thereof joins connectors at the sound output for the coverings being able to receiving sound.

Referring to FIG. 2 further, as soon as the earphone with coverings according to the present invention is worn, the respective projection 11 on the coverings 1 can be plugged into the ears properly. Thus, the sound effect can be enhanced soundly and the external part of ears may be relieved from the pressure of coverings 1. It is appreciated that the earphone of present invention is possible to eliminate the discomfort of ears and avoid the interference of foreign sound. These advantages of the present invention are not possible to reach through the conventional earphone with coverings.

While the invention has been described with reference to preferred embodiment thereof, it is to be understood that modifications or variations may be easily made without departing from the spirit of this invention, which is defined by the appended claims.

What is claimed is:

1. An earphone with coverings, comprising
two opposite coverings, and
a covering connection with two ends, connecting with said two opposite coverings at said two ends, and providing current leads for transmitting signals;
characterized in that a projection extends outward from a lateral side of each of said two coverings facing the ears respectively for plugging into the ears during the earphone being worn, the respective projection having a size corresponding to the external acoustic meatus of the respective ear and providing at least a sound opening so as to transmit the sound into the meatus.

2. The earphone with coverings according to claim 1, wherein the projection provides a loudspeaker therein.

3. The earphone with coverings according to claim 1, wherein the sound opening is disposed at an end of the projection.

4. The earphone with coverings according to claim 1, wherein the sound opening is disposed at a rim of an end on the projection.

5. The earphone with coverings according to claim 1, wherein the respective covering at a rim of an inner side thereof may be provided with soft pad.
